(19) United States
(12) Patent Application Publication
(10) Pub. No.: US 2019/0038558 A1
(43) Pub. Date: Feb. 7, 2019

(54) PLGA/PEI PARTICLES AND METHODS OF MAKING AND USING THE SAME

(71) Applicant: THE UNIVERSITY OF UTAH RESEARCH FOUNDATION, Salt Lake City, UT (US)

(72) Inventors: Sung Wan KIM, Salt Lake City, UT (US); Kwang Suk LIM, Seoul (KR); Young Sook Lee, Salt Lake City, UT (US)

(21) Appl. No.: 16/068,302

(22) PCT Filed: Jan. 5, 2017
(86) PCT No.: PCT/US17/12355

§ 371 Date: Jul. 5, 2018

Related U.S. Application Data
(60) Provisional application No. 62/274,981, filed on Jan. 5, 2016.

(51) Int. Cl. A61K 9/16; A61K 35/28; A61L 27/26; A61L 27/38; A61P 9/10

(52) U.S. Cl. CPC A61K 9/1647; A61K 35/28; A61L 27/26; A61L 2430/20; A61P 9/10; A61L 2400/06; A61L 27/3834

(57) ABSTRACT

Poly(lactic-co-glycolic acid)/polyethylenimine (PLGA/PEI) particles and methods directed to the preparation of PLGA/PEI particles are provided. Methods of using PLGA/PEI particles for the proliferation of stem cells and/or delivery of stem cells are also provided. For example, methods of treating a subject having, or at risk of developing, a cardiovascular disorder can include administering a therapeutically effective amount of stem cell-loaded PLGA/PEI particles to the subject.

### CROSS-REFERENCE TO RELATED APPLICATION

This application claims the benefit of U.S. Provisional Application No. 62/274,981, filed on Jan. 5, 2016, which is hereby incorporated by reference in its entirety.

### STATEMENT REGARDING FEDERALLY SPONSORED RESEARCH

This invention was made with government support under HL065477 awarded by National Institutes of Health. The government has certain rights in the invention.

### TECHNICAL FIELD

The present disclosure relates generally to the preparation of poly(lactic-co-glycolic acid)/polyethylenimine (PLGA/PEI) particles and methods of making and using the same. In particular, the present disclosure relates to the preparation of porous PLGA/PEI particles and methods of making and using the same. More particularly, the present disclosure relates to methods of using PLGA/PEI particles for the proliferation of stem cells and/or delivery of stem cells. Even more particularly, the present disclosure relates to methods of treating cardiovascular disorders by administering PLGA/PEI particles loaded with stem cells (e.g., mesenchymal stem cells) to a subject having or at risk of developing a cardiovascular disorder.

### BACKGROUND

Mesenchymal stem cells (MSCs) have a pluripotent potential to differentiate into various phenotypes, especially after trauma, disease, and/or aging (see M. F. Pittenger, et al., Science, 284 (1999) 143-147; A. Giordano, et al., J Cell Physiol, 211 (2007) 27-35; B. Parekkadan and J. M. Milwid, Annu Rev Biomed Eng, 12 (2010) 87-117; and A. Bagul, et al., Am J Nephrol, 37 (2013) 16-29; each of which is incorporated by reference herein). MSCs are generally hypo-immunogenic and do not express HLA-II (DR) or blood group antigens, and therefore can evade immunologic complications after transplantation (see A. Giordano, et al., J Cell Physiol, 211 (2007) 27-35; B. Parekkadan and J. M. Milwid, Annu Rev Biomed Eng, 12 (2010) 87-117; A. Bagul, et al., Am J Nephrol, 37 (2013) 16-29; and P. Bianco, et al., Stem Cells, 19 (2001) 180-192; each of which is incorporated by reference herein). MSCs can be obtained from a small volume of bone marrow aspiration, compatible with different delivery methods and formulations with stable phenotypes, and made as a standardized cell product (see A. Giordano, et al., J Cell Physiol, 211 (2007) 27-35; B. Parekkadan and J. M. Milwid, Annu Rev Biomed Eng, 12 (2010) 87-117; A. Bagul, et al., Am J Nephrol, 37 (2013) 16-29; and P. Bianco, et al., Stem Cells, 19 (2001) 180-192; each of which is incorporated by reference herein).

However, MSCs must be expanded on a large scale for clinical applications through lengthy ex vivo expansion for 3-4 weeks, which can reduce transfectability of MSCs, increase cost, and risk contamination and/or alteration of cellular properties (see A. Giordano, et al., J Cell Physiol, 211 (2007) 27-35 and P. Bianco, et al., Stem Cells, 19 (2001) 180-192; each of which is incorporated by reference herein). Also, the beneficial effects of adult stem cells, particularly MSCs, administered after organ injury may be primarily mediated, or at least partially mediated, via combined paracrine, endocrine, and/or homing actions (see M. F. Pittenger, et al., Science, 284 (1999) 143-147; A. Giordano, et al., J Cell Physiol, 211 (2007) 27-35; B. Parekkadan and J. M. Milwid, Annu Rev Biomed Eng, 12 (2010) 87-117; A. Bagul, et al., Am J Nephrol, 37 (2013) 16-29; M. Huls, et al., Kidney Blood Press Res, 31 (2008) 104-110; and J. M. Karp, et al., Cell Stem Cell, 4 (2009) 206-216; each of which is incorporated by reference herein).

Transplantation of MSCs by intravenous or intra-arterial infusion can demonstrate a low engraftment rate and homing can vary from less than 1% to about 10% of systemically administered MSCs at 1 week following injection (see B. Parekkadan and J. M. Milwid, Annu Rev Biomed Eng, 12 (2010) 87-117; P. Bianco, et al., Stem Cells, 19 (2001) 180-192; and M. Huls, et al., Kidney Blood Press Res, 31 (2008) 104-110; each of which is incorporated by reference herein). To overcome the above-discussed hurdles to MSC transplantation, various cell delivery systems including microsphere carriers, hydrogels, natural and synthetic scaffolds, and cell-sheets have been considered (see B. E. Strauer and R. Komowski, Circulation, 107 (2003) 929-934; N. Tano, et al., Mol Ther, (2014); Y. Y. Li, et al., Tissue Eng Part A, 20 (2014) 1379-1391; and J. B. McGlohom, et al., J Biomed Mater Res A, 66 (2003) 441-449; each of which is incorporated by reference herein).

Poly(lactic-co-glycolic acid) (PLGA) copolymers, which have been approved by the FDA for diverse applications, have been widely used as biodegradable and biocompatible scaffolds, for example, in the form of either suspensions for cell cultivation or injections for cultivated cells (see M. Ye, et al., J Control Release, 146 (2010) 241-260; T. K. Kim, et al., Biomaterials, 27 (2006) 152-159; and S.-W. Choi, Y. Zhang, et al., J Mater Chem, 22 (2012) 11442-11451; each of which is incorporated by reference herein). Porous PLGA scaffolds can be produced by a porogen leaching method using salts, carbohydrates, and hydrocarbon waxes (see J. B. McGlohom, et al., J Biomed Mater Res A, 66 (2003) 441-449; T. K. Kim, et al., Biomaterials, 27 (2006) 152-159; S.-W. Choi, et al., J Mater Chem, 22 (2012) 11442-11451; and A. G. Mikos, et al., Biomaterials, 14 (1993) 323-330; each of which is incorporated by reference herein). PLGA scaffolds with open pores can afford a large surface area for cell attachment, which may increase cell seeding density, promote cell growth by facilitating mass transport of nutrients and oxygen, and result in improved regenerating and reconstructing potentials (see T. K. Kim, et al., Biomaterials, 27 (2006) 152-159 and S.-W. Choi, et al., J Mater Chem, 22 (2012) 11442-11451; each of which is incorporated by reference herein). Polyethylenimine (PEI) is a potent polymer for gene delivery. However, at high molecular weight (i.e., 25 kDa or above), PEI can be toxic, causing aggregation with erythrocytes (see S. Han, et al., Bioconjug Chem, 12 (2001) 337-345; incorporated by reference herein). PEI_{1.8k} can be less potent but can also be less toxic than high molecular weight PEI.

Ongoing loss of cardiomyocytes, in which apoptotic and necrotic cardiomyocytes are replaced by fibroblasts that form scar tissue, can be one of the early pathological characteristics of myocardial infarction (MI) (see A. Giordano, et al., J Cell Physiol, 211 (2007) 27-35 and Y. Lee, et al., J Control Release, 171 (2013) 24-32; each of which is incorporated by reference herein). This can lead to adverse cardiac remodeling that may cause contractile dysfunction, heart failure, and/or mortality (see A. Giordano, et al., J Cell Physiol, 211 (2007) 27-35 and Y. Lee, et al., J Control Release, 171 (2013) 24-32; each of which is incorporated by reference herein). Transplantation of skeletal myoblasts has been tried as a promising alternative method for the treatment of MI, but it is difficult to obtain donor cells and can be arrhythmogenic (see A. Giordano, et al., J Cell Physiol, 211 (2007) 27-35 and M. Ou, et al., J Control Release, 142 (2010) 61-69; each of which is incorporated by reference herein).

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 is a diagram depicting a modified water/oil/water (W₁/O/W₂) double emulsion solvent evaporation method.

FIG. 2A is a micrograph of a non-porous PLGA/PEI_{1.8k} (NPP) particle, wherein the scale bar equals 200 μm.

FIG. 2B is a micrograph of a porous PLGA/PEI_{1.8k} (PPP) particle, wherein the scale bar equals 200 μm.

FIG. 3A is a scanning electron microscope micrograph of an NPP particle at 250× magnification.

FIG. 3B is a scanning electron microscope micrograph of an NPP particle at 2,000× magnification.

FIG. 3C is a scanning electron microscope micrograph of a PPP particle at 250× magnification.

FIG. 3D is a scanning electron microscope micrograph of a PPP particle at 2,000× magnification.

FIG. 4 is a graph depicting binding affinity of rat MSCs (rMSCs) on non-porous PLGA (NP) particles, porous PLGA (PP) particles, NPP particles, and PPP particles after 24 hours. The relative cell number (%) was measured using a Cell Counting Kit-8 (CCK-8 kit). *P<0.01 vs. NP; #P<0.01 vs. PP; and § P<0.05 vs. NPP.

FIG. 5 is a series of micrographs depicting rMSC proliferation on PPP particles over 5 days at 4× magnification, as indicated.

FIG. 6 is a graph depicting quantification of time-dependent rMSC growth on rMSC-loaded particles. The cell growth rate was measured using a CCK-8 kit for 2 weeks. #P<0.05 vs. NP; ##P<0.01 vs. NP; †P<0.05 vs. PP; ††P<0.01 vs. PP; and § P<0.05 vs. NPP.

FIG. 7A is a series of images depicting in vivo engraftment rate of human MSCs (hMSCs) alone and hMSC-loaded PPP particles at 2 weeks after injections. The images are representative immunohistochemical (IHC) staining images for CD44+ in the left ventricle (LVb) from each group.

FIG. 7B is a graph depicting quantification of percent CD44+ positive cells (mean±SD). *P<0.01 vs. hMSC alone.

FIG. 8 is a series of representative spectral Doppler images of the proximal LAD coronary artery in transthoracic echocardiography.

FIGS. 9A and 9B are a series of graphs depicting time-dependent functional and geometric effects on post-infarct cardiac remodeling at 1 week (1 wk) and 4 weeks (4 wk) after MI. Male Sprague-Dawley (SD) rats, in seven groups, received intramyocardial injections with a total volume of 200 μl right after I/R in the LAD coronary artery. The seven groups, as indicated, were: sham thoracotomy, I/R only, PPP particles alone, hMSCs alone (2×10⁶ hMSCs), 1 mg of PPP particles loaded with 20×10⁵ hMSCs (High group), 1 mg of PPP particles loaded with 10×10⁵ hMSCs (Medium group), and 1 mg of PPP particles loaded with 5×10⁵ hMSCs (Low group). Data represent means±SEM with n=9 per group. FIG. 9A depicts systolic function evaluated by ejection fraction (EF (%)) of the left ventricle (LV). FIG. 9B depicts LV dimension during diastole (LVDd) and systole (LVDs). #P<0.05 vs. sham thoracotomy; *P<0.05 vs. I/R; ‡P<0.05 vs. PPP particles alone; and § P<0.05 vs. hMSCs alone.

FIGS. 10A and 10B are a series of graphs depicting hemodynamic improvements of coronary artery blood flow on post-infarct cardiac remodeling 1 week (1 wk) and 4 weeks (4 wk) after MI in the groups of FIGS. 9A and 9B. FIG. 10A depicts the diameter of coronary artery and total blood volume as measured by velocity time integral of left anterior descending coronary artery (LAD_VTI), as indicated. FIG. 10B depicts coronary artery (CA) output in ml/minute and CA stroke volume (SV) in μL measured at the proximal LAD, as indicated. Data represent means±SEM with n=9 per group. #P<0.05 vs. sham thoracotomy; *P<0.05 vs. I/R; ‡P<0.05 vs. PPP particles alone; and § P<0.05 vs. hMSCs alone.

FIGS. 11A and 11B show that the hMSC-loaded PPP delivery system leads alleviates cardiomyocyte loss and apoptotic activity 4 weeks after MI in the groups of FIGS. 9A and 9B. FIG. 11A is a series of images depicting cardiomyocyte loss as evaluated by IHC staining of cardiomyocyte-specific cardiac troponin T (cTnT) of rat heart myocardium. FIG. 11B depicts representative TUNEL staining images in the border zone of an LV infarct from each group. n=9 per group.

FIGS. 12A and 12B are a series of images illustrating that prolonged engraftment of the hMSC-loaded PPP delivery system induces angiogenesis and suppresses post-infarct fibrosis 4 weeks after MI. FIG. 12A depicts IHC staining images of α-SMA-positive arterioles. FIG. 12B depicts representative Masson's trichrome staining images in the mid-LV of hearts from each group. n=9 per group.

### DETAILED DESCRIPTION

This disclosure is related generally to the preparation of poly(lactic-co-glycolic acid)/polyethylenimine (PLGA/PEI) particles and methods of making and using the same. In particular, the present disclosure relates to the preparation of porous PLGA/PEI particles and methods of making and using the same. More particularly, the present disclosure relates to methods of using PLGA/PEI particles for the proliferation of stem cells and/or delivery of stem cells. Even more particularly, the present disclosure relates to methods of treating cardiovascular disorders by administering PLGA/PEI particles loaded with stem cells (e.g., mesenchymal stem cells) to a subject having or at risk of developing a cardiovascular disorder. It will be readily understood that the embodiments, as generally described herein, are exemplary.

The following more detailed description of various embodiments is not intended to limit the scope of the present disclosure, but is merely representative of various embodiments. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. Each and every reference recited herein is incorporated by reference in its entirety.

A first aspect of the disclosure relates to methods of treating a patient having a cardiovascular disorder. Cardiovascular disorders may include, but are not limited to, myocardial infarction (MI), heart failure, cardiac dysrhythmia, cardiomyopathy (e.g., dilated, hypertrophic, ischemic, uremic, etc.), congenital heart disease, coronary heart disease, coronary artery disease, hypertensive heart disease, diabetic heart disease, inflammatory heart disease, ischemic heart disease, pulmonary heart disease, rheumatic heart disease, and/or valvular heart disease.

In some embodiments, this disclosure provides methods of treating a subject or a patient having a cardiovascular disorder, wherein the methods comprise administering an effective amount of mesenchymal stem cell (MSC)-loaded poly(lactic-co-glycolic acid)/polyethylenimine (PLGA/PEI) particles to a subject or a patient to mitigate or reduce a pathological effect or symptom of or associated with the cardiovascular disorder. In various embodiments, the pathological effects or symptoms may include, but are not limited to: cardiac tissue damage; cardiomyocyte necrosis; contractile dysfunction; cardiac extracellular matrix (ECM) remodeling; cardiomyocyte loss; apoptosis; apoptosis/necrosis; cardiac fibrosis; angiogenesis; hemodynamic and cardiac geometric deterioration in the heart, coronary artery, and/or vascular system; and/or systolic/diastolic dysfunction.

As used herein, “MSC” may refer to any type of mesenchymal stem cell including, but not limited to, human MSCs and/or rat MSCs. For example, MSCs, as used herein, may be extracted from any suitable organism.

As used herein, “PLGA” may refer to any type of poly(lactic-co-glycolic acid) or poly(lactic-co-glycolic acid) copolymer. For example, in some embodiments, PLGA may refer to poly(_{D,L}-lactide-co-glycolide).

As used herein, “PLGA/PEI particle” may refer to a particle or particles wherein the PEI comprises a high molecular weight PEI (e.g., about 25 kDa or above), a medium molecular weight PEI (e.g., about 1.8 kDa to about 25 kDa), a low molecular weight PEI (e.g., about 1.8 kDa or below), or a combination thereof. PLGA/PEI particles may also comprise microparticles, spheres, microspheres, beads, microbeads, and the like. In further embodiments, the PLGA/PEI particles may be porous (PPP) and/or non-porous (NPP). NPP particles, as used herein, may refer to any non-porous PLGA/PEI particles wherein the PEI comprises any molecular weight (e.g., 1.8 kDa, 25 kDa, etc.). PPP particles, as used herein, may refer to any porous PLGA/PEI particles wherein the PEI comprises any molecular weight (e.g., 1.8 kDa, 25 kDa, etc.). In some embodiments, the lactide:glycolide ratio may be about 50:50. In some other embodiments, the lactide:glycolide ratio may be about 30:70, about 40:60, about 60:40, about 70:30, or another suitable ratio. In yet some other embodiments, the lactide:glycolide ratio may be between about 30:70 and about 70:30 or between about 40:60 and about 60:40.

In certain embodiments, the effective amount of stem cell-loaded or MSC-loaded PLGA/PEI particles may be a therapeutically effective amount of the stem cell-loaded or MSC-loaded PLGA/PEI particles. The effective amount, or the therapeutically effective amount, of stem cell-loaded or MSC-loaded PLGA/PEI particles may further comprise a pharmaceutically acceptable carrier.

In some embodiments, the stem cell-loaded or MSC-loaded PLGA/PEI particles may form scaffolds. As discussed above, in certain embodiments, the PEI may comprise a low molecular weight PEI (e.g., PEI_{1.8k}). PEI having other molecular weights is also within the scope of this disclosure and may also be used to prepare the PLGA/PEI particles described herein. In various embodiments, the PLGA/PEI particles and/or the PLGA/PEI_{1.8k} particles may be porous and/or non-porous.

Administration of the stem cell-loaded or MSC-loaded PLGA/PEI particles may be conducted via various methods. For example, the administration may be conducted via an injection such as an intramyocardial injection, a local injection, an intracoronary injection, and/or an intravenous injection. Further, the stem cell-loaded or MSC-loaded PLGA/PEI particles may be injected at or adjacent a fibrotic zone. For example, a fibrotic zone may comprise an area or zone of cardiac tissue wherein apoptotic and/or necrotic cardiomyocytes have been replaced by fibroblasts that form scar tissue. Additionally, administration of stem cell-loaded or MSC-loaded PLGA/PEI particles may be conducted via multiple injections at one or more locations or sites.

The present disclosure also relates to methods of treating or prophylactically treating a subject or a patient at risk of developing a cardiovascular disorder. In some embodiments, this disclosure provides methods of treating or prophylactically treating a patient at risk of developing a cardiovascular disorder, wherein the methods comprise administering an effective amount of stem cell-loaded or MSC-loaded PLGA/PEI particles to reduce a risk of developing the cardiovascular disorder. As described above, the effective amount, or therapeutically effective amount, of stem cell-loaded or MSC-loaded PLGA/PEI particles may also comprise one or more pharmaceutically acceptable carriers. In some embodiments, the stem cell-loaded or MSC-loaded PLGA/PEI particles may form scaffolds. The PEI may comprise a low molecular weight PEI (e.g., PEI_{1.8k}). PEI having other molecular weights is also within the scope of this disclosure and may also be used to prepare the PLGA/PEI particles described herein. In certain embodiments, the PLGA/PEI particles and/or PLGA/PEI_{1.8k} particles may be porous and/or non-porous.

Another aspect of the present disclosure relates to methods of treating a subject or a patient who has had or experienced an MI. The post-MI, or post-infarcted, heart can undergo a series of structural changes termed left ventricular (LV) remodeling. LV remodeling can occur at the organ, cellular, and/or molecular levels. LV remodeling can also occur with three overlapping phases: the inflammatory phase, the proliferative phase, and the healing phase. Initially, cardiac remodeling can be an adaptive response, for example, to maintain substantially normal cardiac function. Cardiac remodeling, however, can gradually become maladaptive or inexorably maladaptive over time (e.g., over subsequent months, years, etc.), and cardiac remodeling can lead to adverse clinical outcomes including heart failure, arrhythmia, and/or mortality.

In some embodiments, the patient may have had an acute MI. This disclosure provides methods of treating the patient who has had an MI, wherein the methods comprise administering an effective amount of stem cell-loaded or MSC-loaded PLGA/PEI particles to mitigate or reduce a pathological effect or symptom of the MI. In various embodiments, the pathological effect or symptom of the MI may include cardiac tissue damage, cardiomyocyte necrosis, cardiac remodeling, LV remodeling, arrhythmia, heart failure, and/or contractile dysfunction.

As discussed above, the effective amount, or the therapeutically effective amount, of stem cell-loaded or MSC-loaded PLGA/PEI particles may also comprise one or more pharmaceutically acceptable carriers.

In certain embodiments, the administration of the stem cell-loaded or MSC-loaded PLGA/PEI particles for the treatment of a subject or a patient who has had an MI may be conducted via various methods. The administration may be conducted via an injection such as an intramyocardial injection. In some embodiments, the stem cell-loaded or MSC-loaded PLGA/PEI particles may be injected at or adjacent a fibrotic zone, as discussed above. In various embodiments, the stem cell-loaded or MSC-loaded PLGA/PEI particles may be injected at or adjacent an infarct. Additionally, administration of stem cell-loaded or MSC-loaded PLGA/PEI particles may be conducted via multiple injections at one or more locations or sites.

The present disclosure also relates to methods of treating a subject or a patient at risk of having or experiencing an MI. In some embodiments, this disclosure provides methods of treating a patient at risk of having an MI, wherein the methods comprise administering an effective amount of stem cell-loaded or MSC-loaded PLGA/PEI particles to reduce the risk of having an MI, as discussed regarding the above methods.

Another aspect of the disclosure relates to methods of making, preparing, manufacturing, or generating PLGA/PEI particles. In some embodiments, the methods of making PLGA/PEI particles may comprise combining PLGA with methylene chloride to generate a PLGA solution. The methods may also comprise diluting PEI in acetone to generate a PEI solution. In certain embodiments, the methods may comprise mixing at least a portion of the PLGA solution with at least a portion of the PEI solution to form or generate a pre-homogenization solution. The pre-homogenization solution may then be homogenized to generate a primary emulsion. In various embodiments, the methods may further comprise the steps of combining the primary emulsion with a polyvinyl alcohol solution to generate a primary emulsion solution and re-emulsifying the primary emulsion solution to generate PLGA/PEI particles.

In some embodiments, the methods of making PLGA/PEI particles may further comprise the step of adding a salt solution (e.g., sodium chloride solution, calcium salt solution, magnesium salt solution, etc.) to at least a portion of the pre-homogenization solution. Addition of the salt solution may form or generate porous PLGA/PEI particles.

In various embodiments, the plurality of PLGA/PEI particles may comprise microparticles, spheres, microspheres, beads, and/or microbeads. Further, as discussed above, the PEI may comprise a high molecular weight PEI (e.g., about 25 kDa or above), a medium molecular weight PEI (e.g., about 1.8 kDa to about 25 kDa), a low molecular weight PEI (e.g., about 1.8 kDa or below), or a combination thereof. Additional methods of preparing PLGA/PEI particles are described in further detail in the Examples section below.

In a further aspect of the present disclosure, PLGA/PEI particles are disclosed. PLGA/PEI particles may comprise PLGA and PEI. The PEI may comprise a high molecular weight PEI (e.g., about 25 kDa or above), a medium molecular weight PEI (e.g., about 1.8 kDa to about 25 kDa), a low molecular weight PEI (e.g., about 1.8 kDa or below), or a combination thereof. Further, the particles may have a diameter of between about 80 μm and about 1000 μm. In other embodiments, the particles may have a diameter of from about 100 μm to about 800 μm. In others, the particles may have a diameter of about 200 μm. In some other embodiments, the particles may have a diameter of between about 200 μm and about 700 μm, between about 300 μm and about 600 μm, between about 400 μm and about 500 μm, or another suitable diameter.

The particles may also comprise pores. For example, each pore may have a diameter of up to about 20 μm. In some embodiments, the diameter of each pore may be between about 1 μm and about 20 μm, between about 5 μm and about 15 μm, between about 8 μm and about 12 μm, or another suitable diameter. In certain embodiments, the PLGA/PEI particles may be loaded with stem cells (e.g., MSCs).

Stem cells, due at least in part to their self-renewing and/or tissue-regenerating properties, may provide therapeutic approaches for various medical needs. Several stem cell populations comprise pluripotent characteristics. Thus, the methods as disclosed herein may also be modified to deliver and/or use various types of stem cells, or adult stem cells.

As described above, the tissue-regenerating properties of MSCs may provide a therapeutic approach for many medical needs, including, but not limited to, adverse post-MI remodeling (see M. F. Pittenger, et al., Science, 284 (1999) 143-147; A. Giordano, et al., J Cell Physiol, 211 (2007) 27-35; B. Parekkadan and J. M. Milwid, Annu Rev Biomed Eng, 12 (2010) 87-117; A. Bagul, et al., Am J Nephrol, 37 (2013) 16-29; M. Huls, et al., Kidney Blood Press Res, 31 (2008) 104-110; L. Armstrong, et al., Stem Cells, 30 (2012) 2-9; and B. E. Strauer and R. Komowski, Circulation, 107 (2003) 929-934; each of which is incorporated by reference herein). Alternative mechanisms, such as paracrine action, may be mediators of tissue protection and regeneration. The beneficial effects of adult stem cells, such as MSCs, administered after organ injury may be primarily mediated, or at least partially mediated, via complex paracrine and endocrine actions, including, but not limited to, amelioration of inflammatory manifestation, modulation of immune response, mitogenics, anti-apoptotic and anti-inflammatory effects, and/or stimulation of vasculogenesis and angiogenesis. Cell therapy can have the capacity to prevent donor cell death and augment the reparative and regenerative effects of cell transfer. MSCs have been applied to treat various diseases including MI, cerebrovascular disease, hind limb ischemia, and ischemic bowel disease. However, as described, the therapeutic efficacy of MSCs can be limited by MSCs' poor survival and short life span, and low engraftment and homing rates of transplanted MSCs. Additionally, in clinical applications, it can take about 1 month to grow a therapeutically sufficient amount of MSCs. To overcome the obstacles to therapeutic use of MSCs, improve MSC life span, and increase MSC engraftment rate, the therapeutic efficacy of MSC-loaded porous PLGA/PEI particles was elucidated, as discussed herein. Further, porous PLGA/PEI particles can work as a supporting system for MSCs (i.e., similar to an extracellular matrix in a living organism).

As described in more detail below, the composition of PLGA particles has been enhanced or improved for use in physiological conditions. To enhance or improve characteristics of PLGA particles, the characteristics of PLGA particles were measured depending, for example, on PLGA molecular weight, ratio between lactic acid and glycolic acid, and PLGA end groups. Particles made of PLGA alone (porous and non-porous), with a size of about 200 μm, were made as described below. The pore size of PP particles was about 20 μm. Further, PLGA 75 kDa (lactide:glycolide ratio 1:1) was used and applied to sequential experiments.

Also, PEI_{1.8k} was mixed with PLGA to improve PLGA's cellular adhesion characteristics and to form PLGA/PEI_{1.8k} particles using methods as discussed herein. In some of the embodiments, each of the PPP particles and NPP particles may be about 200 μm in size; however, other particle sizes are also contemplated. The PPP particles further comprise pores of about 20 μm; however, other pore sizes are also contemplated. PPP particles exhibit higher or improved MSC adhesion on particle surfaces compared with NPP particles and PLGA-only particles. Additionally, PEI_{1.8k} can increase the positive charge of the PLGA surface. As described below, MSCs can survive on PPP particles for 2 weeks in vitro and can be grown from PPP particles. The data, as disclosed herein, suggests that PPP particles may support the enhancement or improvement of MSC survival. Further, PPP particles (e.g., PLGA/PEI_{1.8k} blending polymer based porous particles) can enhance or improve the binding affinity of MSCs in comparison with PLGA-only based particles. Further, PPP particles can also construct or form an anchoring and/or support system for MSC loading. A PPP particle delivery system of the present disclosure may comprise an MSC augmentation effect such that compatible therapeutic effects may be realized using a lower amount or number of MSCs. Cost-effectiveness and time-efficiency may also be improved by the disclosed PPP particle delivery system. For example, methods of the present disclosure may utilize fewer MSCs than some other methods, such that time and cost is decreased (i.e., the culturing of MSCs can be expensive and time-consuming). Decreases in the amount or number of MSCs used can also decrease the risk of infection and avoid or limit loss of MSC pluripotency. Further, a PPP particle delivery system may be applied to MSC delivery for enhancement or improvement of MSC survival rate and/or MSC therapeutic efficacy.

In some embodiments of the present disclosure, injectable PPP microspheres for delivery of MSCs have been developed. Additionally, the characteristics of PPP scaffolds and the potential of PPP scaffolds for the delivery of MSCs have been evaluated, as described in more detail below.

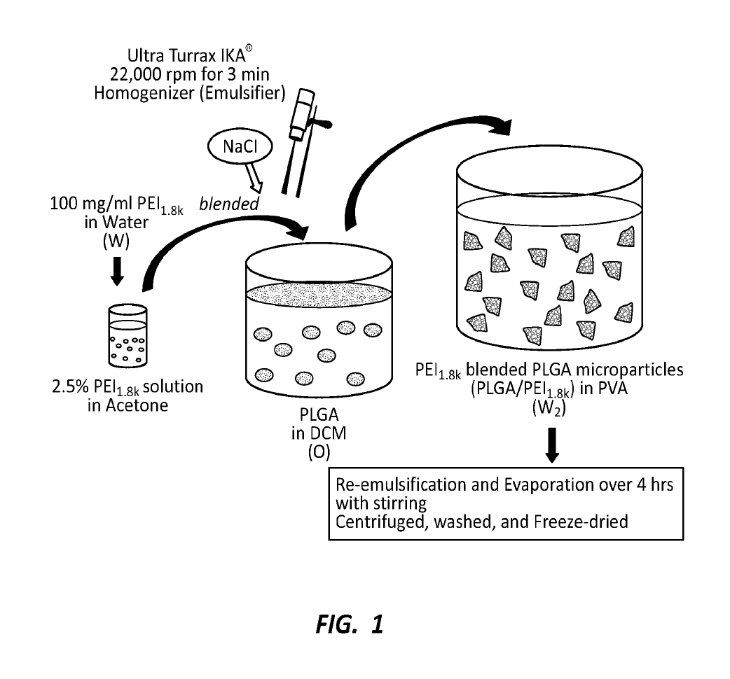

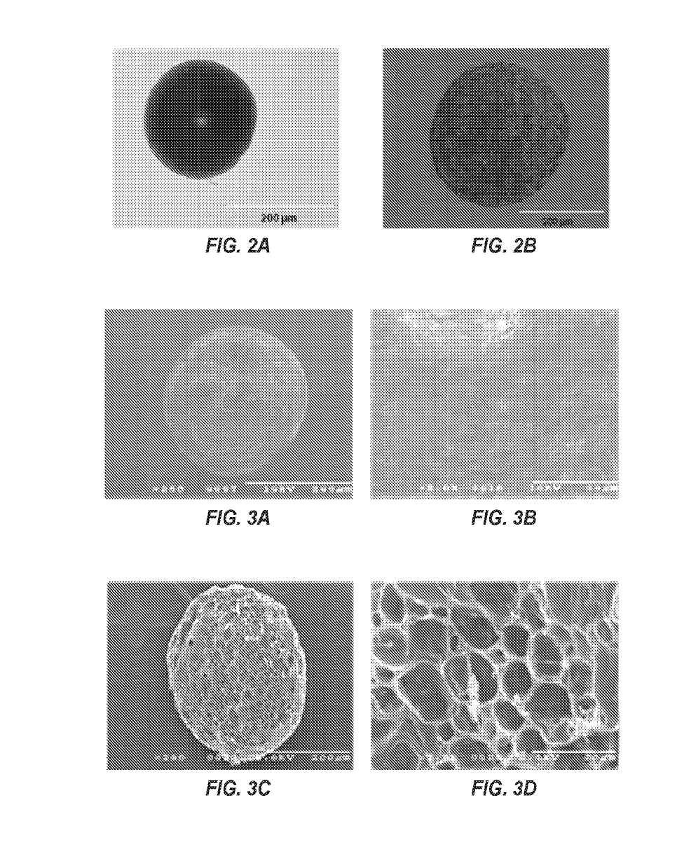

PLGA and PLGA/PEI_{1.8k} microspheres were produced by a W₁/O/W₂ double emulsion solvent evaporation method using sodium chloride as a salting-out agent, which formed large, isolated, and scattered pores in the inner region of the PLGA and PLGA/PEI_{1.8k} microspheres due, at least in part, to the immediate, or substantially immediate, coalescence of the aqueous droplets during the solvent removal (see T. K. Kim, et al., Biomaterials, 27 (2006) 152-159; incorporated by reference herein). Shown in FIG. 1 is a diagram depicting a modified water/oil/water (W₁/O/W₂) double emulsion solvent evaporation method. The oil phase was methylene chloride comprising PLGA and acetone comprising PEI_{1.8k}. Both NP and PP microspheres did not appear to affect the binding affinity of rMSCs. To potentially improve the binding affinity of the rMSCs to the microparticles, PEI_{1.8k} blended PLGA (PLGA/PEI_{1.8k}) was introduced. Without being bound by theory, it was hypothesized that the positive charge of PEI_{1.8k} could increase the binding affinity of the rMSCs to the PLGA/PEI_{1.8k} microparticles. PEI₂₅ₖ has a higher positive charge than PEI_{1.8k} in aqueous solutions and is a notable non-viral vector. Although PEI_{1.8k} showed lower transfection efficiency than PEI₂₅ₖ, PEI_{1.8k} comprises a positive charge and high biocompatibility. FIGS. 2A and 2B are micrographs of an NPP particle and a PPP particle, respectively, wherein the scale bar equals 200 μm. As depicted in FIG. 2B, the formation of open pores in the interior region of a PPP particle can be seen via an optical microscope as light passing through the particle. The PPP particles produced by the method described above demonstrated an average particle size of 200 μm (see FIGS. 2A, 2B, and 3A-3D). Using 5% sodium chloride solution, surface pores of up to 20 μm in diameter were produced in the PPP particles (see FIGS. 3A-3D).

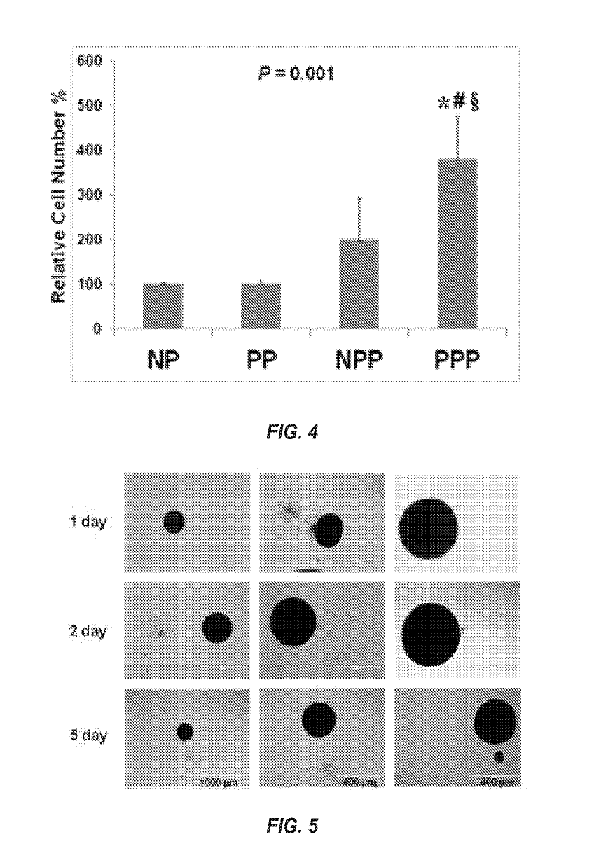

The characteristics of PPP microspheres as a suspension micro-carrier for rMSCs and hMSCs were evaluated. After inoculation of PPP particles mixed with rMSCs in a 24-well plate for 24 hours, the PPP particles showed higher binding affinity to rMSCs compared with other particle groups (P=0.001; see FIG. 4). In contrast, the binding affinity of NP and PP microspheres was not significant (see FIG. 4). The PPP scaffold showed approximately 4 times and approximately 2 times higher binding affinity to rMSCs than PLGA particles (P<0.01; NP and PP) and NPP particles (P<0.05), respectively (see FIG. 4). Both the structural entrapment on the surface pores of the microspheres and the physical conjugation with the positive charge of PEI_{1.8k} in the PPP microspheres may, at least in part, be mechanisms that result in the enhanced binding affinity as observed. These results can suggest that microparticles with open pores may provide a favorable, or more favorable, spatial environment for the attachment and delivery of MSCs.

After attachment of rMSCs to PPP microparticles, the rMSC-loaded PPP microparticles were moved into a new plate with fresh Dulbecco's modified Eagle's medium (DMEM) comprising 10% fetal bovine serum (FBS). Growth of the rMSCs from the PPP microparticles was imaged up to 5 days (see FIG. 5). This result indicates that PPP particles may play a role as a favorable, or more favorable, scaffold for the growth of MSCs.

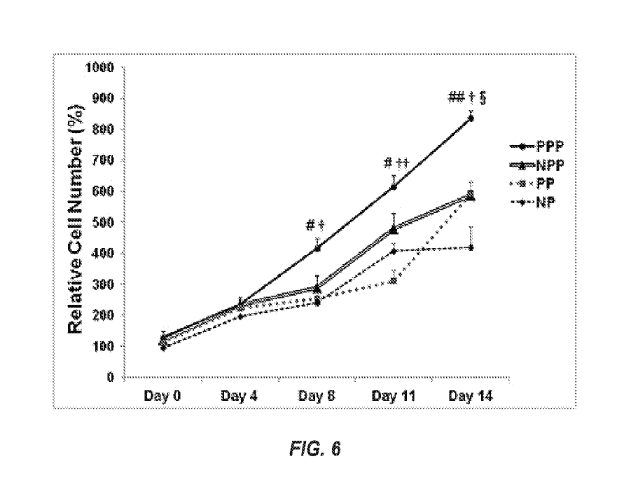

Next, the time-dependent growth rate of rMSCs anchored on the rMSC-loaded microparticles was quantified using a CCK-8 kit for 2 weeks (see FIG. 6). The proliferation rate of rMSCs on PPP microspheres was significantly higher than in the PLGA groups (NP and PP) from 8 days after cultivation (P<0.05 on day 8; P<0.01 on day 11; see FIG. 6). At 14 days after cultivation, the proliferation rate of rMSCs on PPP particles was more prominent than the PLGA (NP and PP) and NPP groups (P<0.01; see FIG. 6). This result indicates that PPP scaffolds may provide a more biocompatible microenvironment for the proliferation of MSCs.

The positivity of CD34− in hMSC was measured at below 2% in flow cytometry analysis, and the positivity of CD34− in hMSC was independent of the concentration of CD34 antibody. The positivity of CD44+ in hMSC was measured at over 95% in flow cytometry analysis, and the positivity of CD44+ in hMSC was independent of hMSC cell number. The positivity of CD44+ in hMSC was affected by the concentration of CD44 antibody per well. The newly grown hMSCs attached to the plate surface (excluding floating hMSC-loaded PPP particles) were compared with the hMSC-only group and demonstrated a relative CD44+ positivity of 61.5%. Therefore, the hMSC loading capacity of floating hMSC-loaded PPP particles was estimated at approximately a maximum of 38.5%.

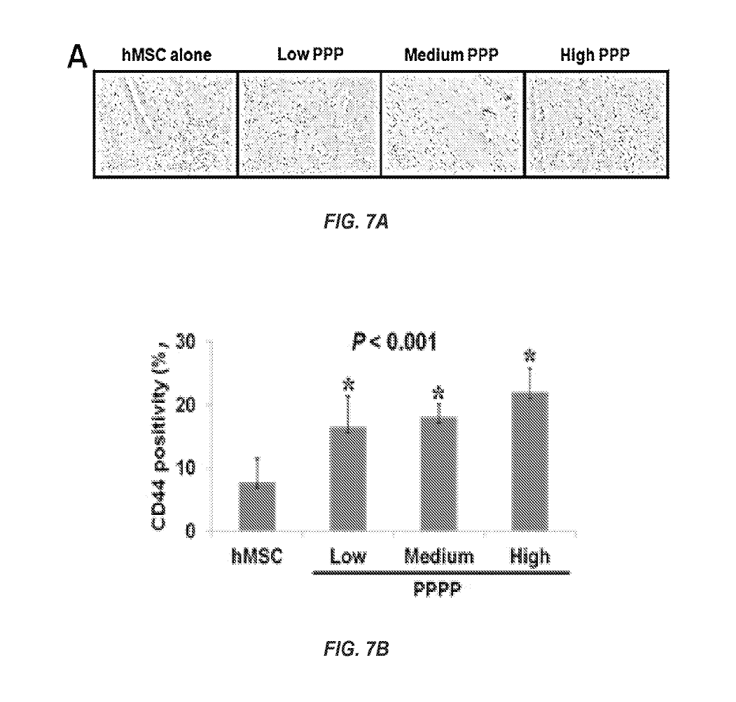

To evaluate the in vivo engraftment rate of hMSC-loaded PPP particles, hMSCs alone and three different amounts of hMSC-loaded PPP particles were administered via intramyocardial injection after MI into rats. The three different amounts of hMSC-loaded PPP particles all doubled the engraftment rate compared with hMSCs alone in rats (P<0.001; see FIGS. 7A and 7B). The engraftment rate of hMSC between the three different amounts of hMSC-loaded PPP groups was comparable. In this result, when 1 mg of the PPP amount was fixed per rat, even the 5×10⁵ hMSC-loaded PPP (Low) group appeared to saturate the hMSC loading capacity. The significance of in vivo engraftment rates of the three different hMSC-loaded PPP groups may be further elucidated using in vivo functional and histopathologic analyses.

Although bone marrow MSCs (BM-MSCs) can be easily harvested from bone marrow (BM), prolonged ex vivo culture time can limit their therapeutic efficacy following transplantation into humans because the prolonged ex vivo culture time can reduce the potential of BM-MSCs to differentiate. For example, donor BM-MSC migration into damaged tissue is generally not sufficient and the ability of BM-MSCs to transdifferentiate may be limited (i.e., only 10-20%). The PPP group loaded with 5×10⁵ hMSCs demonstrated approximately twice the in vivo engraftment rate of the hMSCs alone (20×10⁵ hMSCs) at 2 weeks after MI. These results may suggest that using only 25% of the current amount of hMSCs could result in greater regenerative potential. This PPP delivery system may provide improved survival and engraftment after hMSC transplantation, shortened ex vivo time, and/or lowered costs, while substantially maintaining the characteristics of hMSCs. After transplantation, MSCs can act in both endocrine and paracrine pathways, and MSCs are considered an attractive vehicle in cell therapy (see B. Parekkadan and J. M. Milwid, Annu Rev Biomed Eng, 12 (2010) 87-117 and T. Meyerrose, et al., Adv Drug Deliv Rev, 62 (2010) 1167-1174; each of which is incorporated by reference herein). Therefore, stem cell-loaded or MSC-loaded PPP delivery may be effective as a dual-scaffold system that results in pathophysiologic and functional improvements. PPP may also be a promising injectable scaffold for the delivery of hMSCs to repair tissue.

### EXAMPLES

The following examples are illustrative of disclosed methods and compositions. In light of this disclosure, those of skill in the art will recognize that variations of these examples and other examples of the disclosed methods and compositions would be possible without undue experimentation.

### Example 1—Materials

Poly(_{D,L}-lactide-co-glycolide) (lactide:glycolide ratio 50:50, MW 80,000 Da) (PLGA) was purchased from Polyscitech® (West Lafayette, Ind.). Polyethylenimine (MW 1,800 Da, PEI_{1.8k}) and polyvinyl alcohol (PVA, 87-89% hydrolyzed, MW 31,000 Da-50,000 Da) were purchased from Sigma-Aldrich® (St. Louis, Mo.). DMEM, Dulbecco's phosphate buffered saline (DPBS), phosphate buffered saline pH 7.4 (PBS, pH 7.4), and fetal bovine serum (FBS) were purchased from Invitrogen™ (Carlsbad, Calif.). All others reagents were of analytical grade.

### Example 2—Preparation of PPP Particles

Porous microspheres were prepared by a modified W₁/O/W₂ double emulsion solvent evaporation method (see, e.g., FIG. 1). 250 mg of PLGA was dissolved in 10 ml of methylene chloride (DCM). 125 μl of PEI_{1.8k} in distilled water (100 mg/ml) was diluted in 4.875 ml of acetone, making a 2.5% working concentration (2.5% v/v). 250 μl of prepared 2.5% PEI_{1.8k} solution was blended with 2 ml of 25 mg/ml PLGA solution and then stirred at 400 rpm for 30 minutes. To produce PP particles and PPP particles, 250 μl of 5% sodium chloride solution was added to a PLGA/PEI_{1.8k} solution. In some preparations, the PPP particles had an average particle size of 290 μm and an average pore size of 14.3 μm.

NP particles and NPP particles were produced without adding the above-mentioned salts (e.g., sodium chloride). The first W₁/O emulsion was prepared using a homogenizer (Ultra Turrax IKA® T18 basic; IKA® Works Inc., Wilmington, N.C.) at 22,000 rpm for 3 minutes. This primary emulsion was immediately poured into 400 ml of 0.4% (w/v) PVA solution and then was re-emulsified using stirring at 400 rpm overnight. After the solvent was evaporated, the microparticles were separated by centrifugation, and washed three times with distilled water. Then, the microparticles were lyophilized using a freeze dryer. The particles made in one batch were combined with 2 ml of distilled water and used for eight rats (250 μl/rat, 250 μl/96-well plate).

### Example 3—Characterization of PPP Microspheres

PPP microparticles were visualized using an optical microscope with a digital camera. Then, the gross morphology and the pores of the particles were detected by scanning electron microscopy (Hitachi™ S-3000N). The average diameter and size distribution of the microspheres were determined by scanning electron microscopy. To determine the surface pore size of the microspheres, five microspheres were analyzed using image software (ImageJ, US National Institutes of Health). The average surface pore size of the microspheres was measured in five microspheres (pores of <5 μm were excluded).

### Example 4—Preparation of MSCs

Rat MSCs (rMSCs) were obtained from American Type Culture Collection (ATCC®, Manassas, Va.). Human MSCs (hMSCs), transferred from the Pharmicell™ Co., Ltd. (Sungnam, South Korea), were used. The hMSCs were isolated from BM aspiration in healthy adult male donors with informed consent. Briefly, the BM aspirate was diluted with phosphate buffered saline (PBS) and then layered over Ficoll® liquid by density gradient centrifugation. Mononuclear cells were placed into a 75 cm² flask and cultivated in low-glucose DMEM containing 10% FBS and 20 μg/ml gentamicin in a humidified incubator at 37° C. under 5% CO₂ for 5 to 7 days. The medium was changed to remove non-adherent cells. The hMSCs displayed a fibroblast-like spindle-shaped appearance, and were characterized by their ability to adhere to plastic in standard culture conditions and to form colony forming units in every passage. When these primary cultures of MSCs reached 80% confluence, the cells were trypsinized and subcultured. The procedure was repeated for continuous maintenance of the cells. For experiments, the expanded cells were stored in liquid nitrogen. The cryopreserved cells were thawed and used for this study. The hMSCs were characterized by flow cytometry (BD Biosciences™), using specific positive surface markers (CD105 and CD73), while being negative for hematopoietic markers such as CD34, CD45, and CD14.

### Example 5—Cell Culture

Dry NP, PP, NPP, and PPP microspheres were sterilized by soaking them in 70% ethanol at 4° C. for 4 hours, and then washing them with PBS (pH 7.4). Subsequently, 2×10⁵ rMSCs were added to a culture media comprising 1 mg/ml of microspheres in 96-well plates. rMSCs were attached to microspheres in a 37° C. incubator under 5% CO₂ with continuous agitation using DMEM supplemented with 20% (v/v) FBS, 100 unit/ml penicillin, and 100 μg/ml streptomycin for 24 hours.

### Example 6—Cell Attachment and Proliferation Analysis

To generate rMSC-loaded microparticles, 1 mg/ml of PLGA particles (NP and PP) and PLGA/PEI_{1.8k} particles (NPP and PPP) were incubated with rMSCs in 96-well plates at 37° C. under 5% CO₂ for 24 hours. The floating rMSC-loaded PPP particles were moved into a new 24-well plate to measure the amount of rMSCs on the PPP microspheres. Cell number was measured by MTT following manufacturer's protocol.

Next, to quantify the time-dependent growth rate of rMSCs anchored on the rMSC-loaded particles for 2 weeks, cell proliferation was evaluated using a Cell Counting Kit (CCK-8, Dojindo Molecular Technologies™, Inc., Rockville, Md.), which determined the number of viable cells in cell proliferation. To evaluate rMSC growth, rMSC-loaded microparticles were moved to a new plate every 3 to 4 days.

To generate hMSC-loaded microparticles, hMSCs and PPP particles were incubated for 24 hours using continuous agitation at 37° C. under 5% CO₂ to attach the cells. The next day, the different amounts of hMSC-loaded PPP particles were administered in rats.

### Example 7—Pathological Analysis

Serial 4 μm thick sections of rat myocardium were fixed, embedded, and stained with H&E stain. Fibrosis, determined by collagen contents, was evaluated by Masson's trichrome stain. Immunohistochemical (IHC) staining was performed on the 4 μm thick sections of formalin-fixed, paraffin-embedded rat heart tissue. Sections were air-dried at room temperature and then placed in a 60° C. oven for 30 minutes to melt the paraffin. All of the staining steps were performed at 37° C. using an automated immunostainer (BenchMark® XT, Ventana Medical Systems®). To evaluate the loss of cardiomyocytes and angiogenesis, determined by the arteriolar density after MI, heart sections were IHC stained using α-smooth muscle actin (α-SMA) and cardiomyocyte-specific troponin T (cTnT). The sections were detected using the ultraView DAB™ detection kit (Ventana Medical Systems®). The sections were counterstained with hematoxylin for 8 minutes. Also, apoptosis by TUNEL positivity in the infarct border zones was evaluated as the number of terminal deoxynucleotidyl transferase (TdT)-labeled nuclei per a unit area. Analysis of all images was randomly chosen within the infarct border zone of LV and carried out in five random high-power fields per section using ImageScope™ (Aperio Technologies® Inc., Vista, Calif.).

### Example 8—Estimation of In Vitro Loading Capacity of hMSC-Loaded PPP Particles

1×10⁵ cells/well of hMSCs in 200 μl DMEM containing 10% FBS were mixed with 1 mg/ml of PPP in 96-well plates. The hMSCs and PPPs were incubated at 37° C. under 5% CO₂ for 24 hours to permit cell attachment. The next day, the floating hMSC-loaded PPPs were collected and moved into a new 24-well plate. After 7 days, the floating cells and particles were removed. The newly expanded hMSCs (attached in the 24-well plate) were evaluated to identify the hMSCs' immunophenotype with CD44+(Santa Cruz Biotechnology™, SC-18849) and CD 34− (Santa Cruz Biotechnology™, SC-7324). After the removal of the floating hMSC-loaded PPP microparticles, the remaining amount of CD44+ hMSCs was compared with the amount of CD44+ cells in the hMSC-only group without particles to estimate the loading capacity of the PPP particles.

### Example 9—In Vivo Engraftment Rate of hMSCs in MI Model

MI was induced in 7-8-week-old male Sprague-Dawley (SD) rats (200-250 g) by surgical occlusion of the left anterior descending (LAD) coronary artery as previously described (see Y. Lee, et al., J Control Release, 171 (2013) 24-32; incorporated by reference herein). Briefly, under mechanical ventilation, the LAD coronary artery was ligated for 30-minute occlusion. Following successful ischemia-reperfusion (I/R), the animals were assigned to one of six groups: sham thoracotomy, I/R only, injection of hMSCs alone, and injection of hMSC-loaded PPP particles (High, Medium, and Low). In the hMSC-alone group, 20×10⁵ hMSCs were administered. The hMSC-loaded PPP particle groups were administered with three different hMSC amounts: 20×10⁵ (High group), 10×10⁵ (Medium group), and 5×10⁵ (Low group). The amount of PPP was fixed at 1 mg per rat. After reperfusion, the rats received a total injection volume of 200 μl delivered to four separate intramyocardial sites with three injections to the border zone of the infarct in left ventricle (LVb) and one injection to the fibrotic central zone in left ventricle (LVc).

In some examples, MI was induced in 7-8-week-old male SD rats (220-250 g) by 30-minute surgical occlusion of the LAD coronary artery. These animals were assigned to one of seven groups (each n=9): sham thoracotomy, I/R only, injection of PPP particles alone, injection of hMSCs alone (2×10⁶ hMSCs), and injection of PPP particles loaded with three different amounts of hMSCs. The hMSC-loaded PPP particle groups were administered with three different hMSC amounts at 1 mg of PPP particle: 20×10⁵ (High group), 10×10⁵ (Medium group), and 5×10⁵ (Low group). After ischemia-reperfusion (I/R), the rats received a total injection volume of 200 μl delivered to four separate intramyocardial sites with three injections to the ischemic border zone of the infarct in LV (LVb) and one injection to the fibrotic central zone of the infarct in LV (LVc) with a 23 ¼ gauge needle. Animals were followed for 4 weeks after intramyocardial transplantation.

IHC staining was performed on the 4 μm thick sections of formalin-fixed, paraffin-embedded rat heart tissue. Sections were air-dried at room temperature and then placed in a 60° C. oven for 30 minutes to melt the paraffin. All of the staining steps were performed at 37° C. using an automated immunostainer (BenchMark® XT, Ventana Medical Systemse). To evaluate the in vivo engraftment rate of hMSC after MI, heart sections were IHC stained using CD44+(mouse anti-pan CD44 monoclonal antibody; Millipore™, Billerica, Mass.; #MAB4065; 1:6,000) and CD34-(CONFIRM™ anti-CD34 (QBEnd/10) Primary Antibody; #790-2927; Ventana Medical Systems®, Tucson, Ariz.; optimally pre-dilute antibody). The sections were detected using the ultraView DAB™ detection kit (Ventana Medical Systems®). The sections were counterstained with hematoxylin for 8 minutes. Analysis of all images was carried out with an ImageScope™ (Aperio® Technologies, Vista, Calif.) and randomly chosen within the LVb by an investigator blinded to the treatment groups. Cells positive for CD44 and CD34 over the infarcted zone were counted in five random high-power fields (20× magnification), using an ImageScope™, per whole heart specimen. Counts from 20 microscopic fields were averaged and expressed as the percent (%) of positivity per high-power field.

### Example 10—Echocardiography

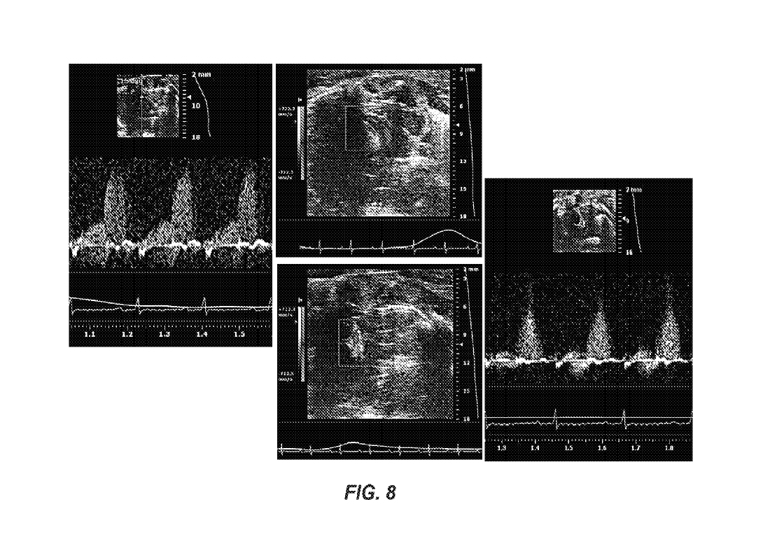

To assess LV remodeling and function in rats, transthoracic echocardiography was performed on weeks 1 and 4 after the intramyocardial administration in rats lightly anesthetized with isoflurane at 1-2 L/minute and spontaneous respiration. Echocardiograms were performed with a special small animal echocardiography system (Vevo2100® High-Resolution Imaging System, VisualSonics™ Inc.) equipped with a 13- to 24-MHz linear-array transducer (MS250, MS400 MicroScan™ Transducer, VisualSonics™ Inc.). Transthoracic coronary blood flow velocity in the proximal LAD coronary artery, infarct-related coronary artery was measured during diastole and systole 1 and 4 weeks after post-infarct intramyocardial injections (see FIG. 8). All measurements were averaged for three consecutive cardiac cycles.

### Example 11—hMSC Delivery System-Associated Improvement of LV Systolic Function and Preservation of Cardiac Geometry

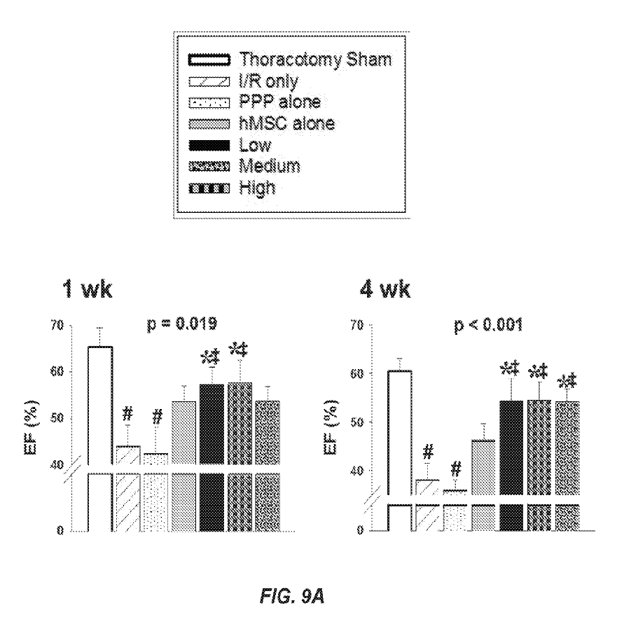

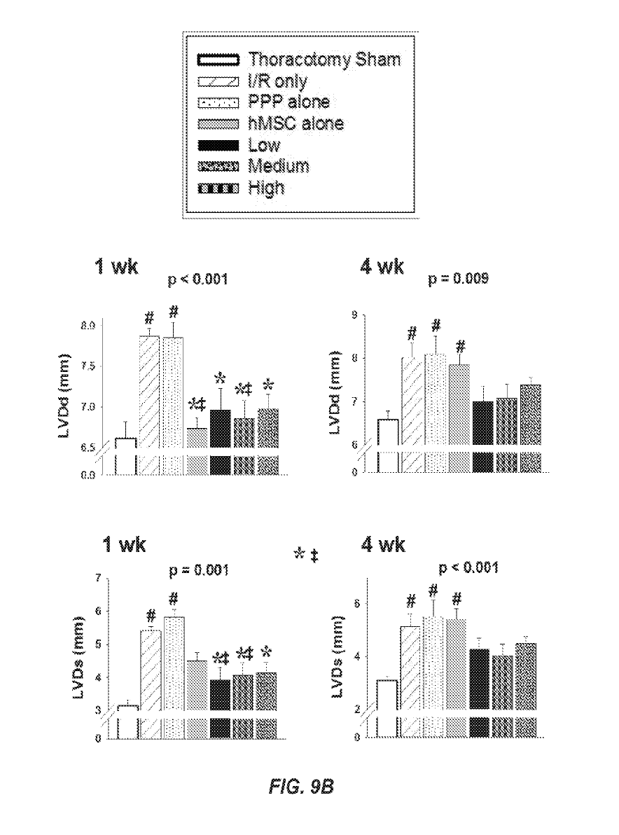

To examine whether delivery of hMSC-loaded PPP microparticles affects time-dependent functional and geometric ischemic cascades in heart, transthoracic echocardiography was performed 1 and 4 weeks after MI. On post-infarct week 1, the administration of both hMSCs alone and hMSC-loaded PPP microparticles showed an improved left ventricular ejection fraction (LVEF) and the LV diameter during the systolic and diastolic phases was comparable to the sham thoracotomy group (see FIGS. 9A and 9B). However, these functional and geometric improvements were sustained only in the hMSC-loaded PPP group at 4 weeks after MI (see FIGS. 9A and 9B). The post wall thickness, interventricular septum thickness, and LV mass during the systolic and diastolic phases did not reveal any differences between the groups at both 1 and 4 weeks after MI. All of the echocardiographic parameters of the PPP-alone injection group were comparable to the I/R-only group, excluding the impact of the PPP microparticles themselves.

### Example 12—hMSC Delivery System-Associated Augmentation of Blood Flow of the Coronary Artery

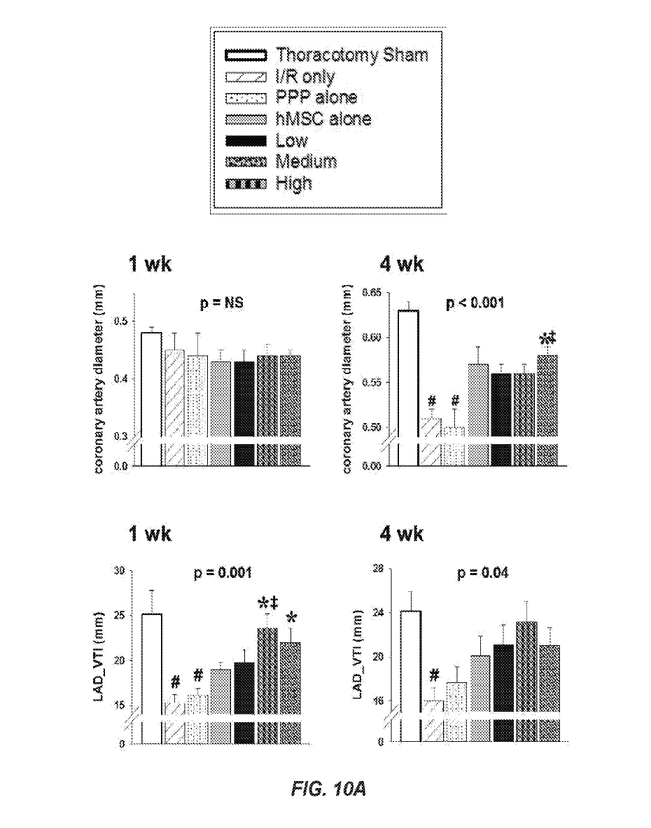

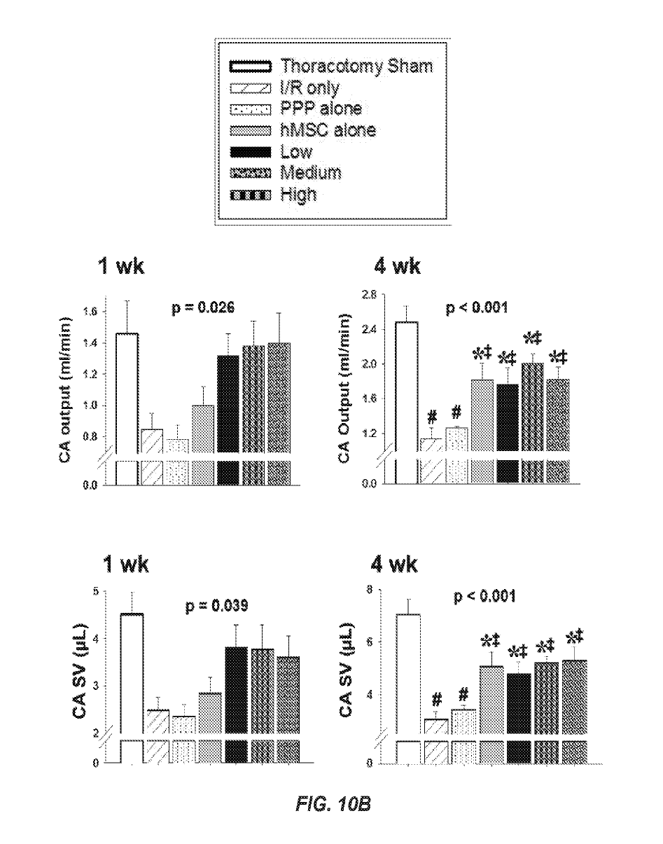

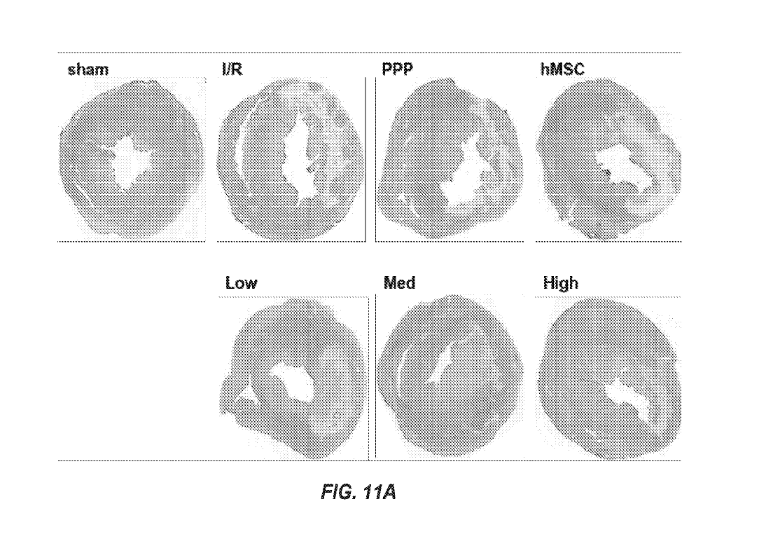

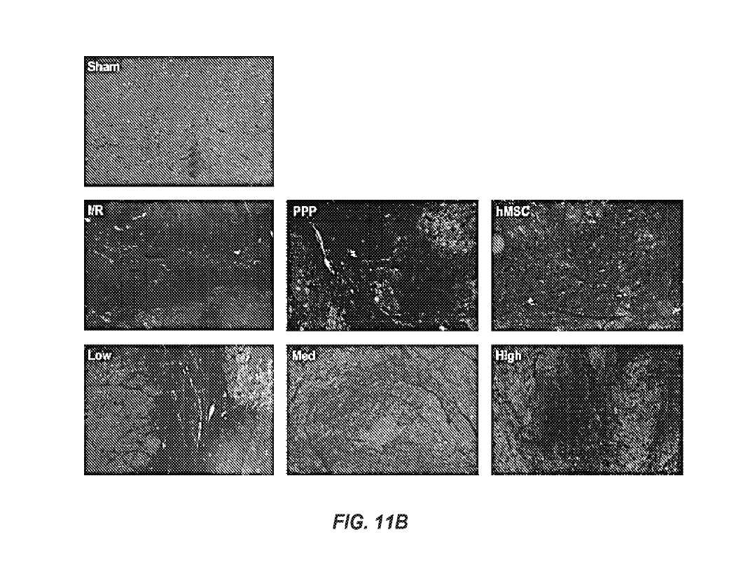

Coronary microvascular function was evaluated by transthoracic Doppler echocardiography (see P. G. Camici and F. Crea, N Engl J Med, 356 (2007) 830-840; T. P. van de Hoef, et al., Circ Cardiovasc Interv, 6 (2013) 207-215; F. Fang, et al., Int J Cardiol 176 (2014) 80-85; and L. Cortigiani, et al., J Am Soc Echocardiogr, 27 (2014) 742-748; each of which is incorporated by reference herein). The blood flow of the proximal left anterior descending (LAD) coronary artery showed a characteristic biphasic blood flow pattern with a larger diastolic component and a smaller systolic component in spectral Doppler echocardiography (see FIG. 8). The hMSC-loaded PPP delivery system demonstrated increased diameter, total blood volume (measured by velocity time integral, VTI), output, and stroke volume of coronary artery at post-infarct week 4 (see FIGS. 10A and 10B). Without being bound to any one theory, these findings may suggest improvements in total blood volume, output, and stroke volume of the coronary artery by the hMSC-loaded PPP microparticle system that may be a potential mechanism to reversing post-infarct cardiac remodeling. This may elucidate, in part, the beneficial effect of the hMSC-loaded PPP delivery system on improving coronary microvascular dysfunction during post-infarct cardiac remodeling over time.

### Example 13—hMSC Delivery System-Associated Decreases in Cardiomyocyte Loss and Apoptotic Activity

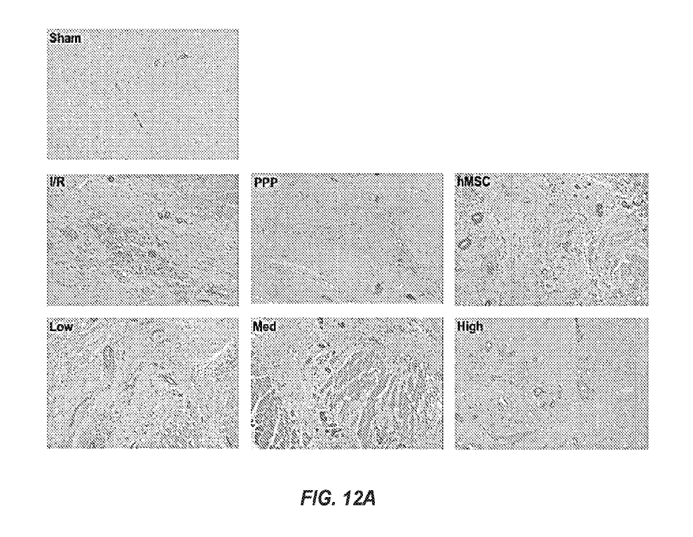

IHC staining of cardiomyocyte-specific cardiac troponin T (cTnT) of the rat heart myocardium was performed 4 weeks after MI. The cardiomyocyte is a major cardiac cell involved in the cardiac remodeling process. The loss of cardiomyocytes after MI is an early distinctive pathologic finding. Compared with the VR, PPP particle-alone, and hMSC-alone groups, the hMSC-loaded PPP groups showed significantly decreased cardiomyocytes loss (see FIG. 11A). Also, post-infarct cardiac remodeling contains diverse cellular changes, including apoptosis. Thus, the effect on apoptotic activity in the border zone of LV infarct was evaluated between the groups. The apoptotic activity measured by TUNEL staining revealed lower apoptosis in the hMSC-loaded PPP group than that of the other groups (see FIG. 11B).

### Example 14—hMSC Delivery System-Associated Enhancement of Angiogenesis and Amelioration of Cardiac Fibrosis with a Reduction in Infarct Size

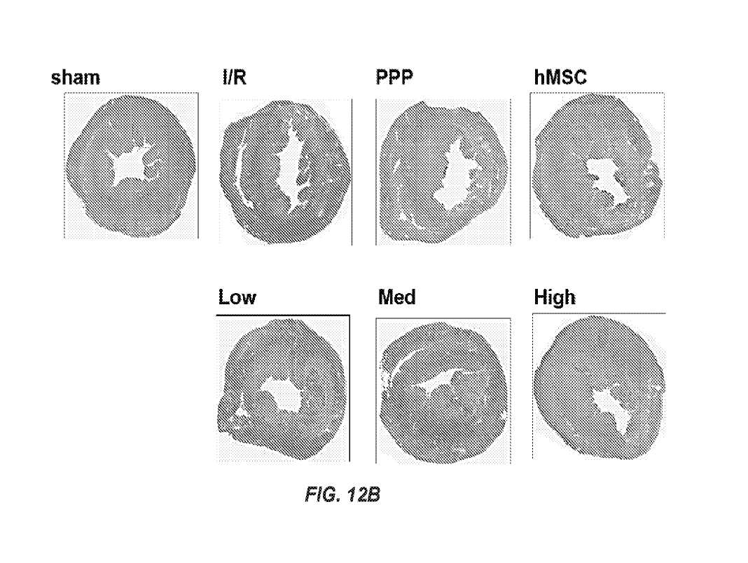

To recover cardiac function after MI, angiogenesis can establish blood supply to infarcted myocardium during the healing phase of post-infarct cardiac remodeling. IHC staining for α-SMA demonstrated more abundant arterioles in the hMSC-loaded PPP particles injection group than in the other treatment groups (see FIG. 12A), suggesting higher upregulation of angiogenic activity in the border zone of the infarct and potentially enhanced oxygen supply to the infarcted myocardium. Fibrosis is also a common final pathological finding resulting from diseases of the heart and other organs. The loss of myocardial muscle mass caused by fibrotic scar formation is related to heart failure, the most common post-infarct morbidity. It was evaluated whether the intramyocardial injections of hMSC-loaded PPP particles had an effect on the suppression of cardiac fibrosis in post-infarct cardiac remodeling. In Masson's trichrome staining of collagen, the post-infarct fibrotic scar areas in the LV were decreased in the hMSC-loaded PPP particles injection group compared to I/R, PPP particle-alone, and hMSC-alone groups (see FIG. 12B). In light of this study, and without being bound by theory, it is hypothesized that the extended stability of hMSC loaded on PPP microparticles in the ischemic cardiac tissue exerts prolonged paracrine effects, enough to reverse the functional, geometric, hemodynamic, and pathologic remodeling process after MI.

### Example 15—Statistical Analysis

Statistical calculations were carried out using SPSS 19.0 software (SPSS™ Inc., Chicago, Ill.). Unless otherwise indicated, data were expressed as the mean±SD. Comparisons between multiple groups were performed by analysis of variance (ANOVA) followed by Tukey post-hoc testing. Groups with P values less than 0.05 were considered statistically significant.

References to approximations are made throughout this specification, such as by use of the term “about.” For each such reference, it is to be understood that, in some embodiments, the value, feature, or characteristic may be specified without approximation. For example, where qualifiers such as “about” and “generally” are used, these terms include within their scope the qualified words in the absence of their qualifiers. For example, where the term “about 200 μm” is recited with respect to a feature, it is understood that in further embodiments, the feature can be precisely 200 μm.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

It will be apparent to those having skill in the art that many changes may be made to the details of the above-described embodiments without departing from the underlying principles of the invention. The scope of the present invention should, therefore, be determined only by the following claims.

1. A method of treating a subject having, or at risk of developing, a cardiovascular disorder, comprising:
administering to the subject a therapeutically effective amount of stem cell-loaded poly(lactic-co-glycolic acid)/polyethylenimine (PLGA/PEI) particles to reduce a pathological effect or symptom associated with the cardiovascular disorder, or to reduce a risk of developing the cardiovascular disorder.

2. The method of claim 1, wherein the stem cells comprise mesenchymal stem cells.

3. The method of claim 1 or claim 2, wherein the particles are selected from at least one of microparticles, microbeads, or microspheres.

4. The method of any one of claims 1-3, wherein the stem cell-loaded PLGA/PEI particles form scaffolds.

5. The method of any one of claims 1-4, wherein the PEI comprises a low molecular weight PEI.

6. The method of claim 5, wherein the PEI comprises PEI_{1.8k}.

7. The method of claim 6, wherein the PLGA/PEI_{1.8k} particles are porous.

8. The method of any one of claims 1-6, wherein the PLGA/PEI particles are porous.

9. The method of any one of claims 1-8, wherein the pathological effect or symptom is selected from at least one of cardiac tissue damage; cardiomyocyte necrosis; contractile dysfunction; cardiac extracellular matrix remodeling; cardiomyocyte loss; apoptosis; necrosis; cardiac fibrosis; angiogenesis; hemodynamic and cardiac geometric deterioration in the heart, coronary artery, or vascular system; or systolic/diastolic dysfunction.

10. The method of any one of claims 1-9, wherein the cardiovascular disorder is selected from at least one of myocardial infarction, heart failure, cardiac dysrhythmia, cardiomyopathy, congenital heart disease, coronary heart disease, coronary artery disease, hypertensive heart disease, diabetic heart disease, inflammatory heart disease, ischemic heart disease, pulmonary heart disease, rheumatic heart disease, or valvular heart disease.

11. The method of any one of claims 1-10, wherein the administration of the stem cell-loaded PLGA/PEI particles is via at least one of an intramyocardial injection, a local injection, an intracoronary injection, or an intravenous injection.

12. The method of claim 11, wherein the particles are injected at or adjacent at least one of a border zone of an infarct, a fibrotic zone of an infarct, or a central fibrotic zone of an infarct.

13. A method of treating a subject who has had a myocardial infarction (MI), or who is at risk of having an MI, comprising:
administering to the subject a therapeutically effective amount of stem cell-loaded poly(lactic-co-glycolic acid)/polyethylenimine (PLGA/PEI) particles to reduce a pathological effect or symptom of the MI, or to reduce the risk of having an MI.

14. The method of claim 13, wherein the stem cells comprise mesenchymal stem cells.

15. The method of claim 13 or claim 14, wherein the particles are selected from at least one of microparticles, microbeads, or microspheres.

16. The method of any one of claims 13-15, wherein the stem cell-loaded PLGA/PEI particles form scaffolds.

17. The method of any one of claims 13-16, wherein the PEI comprises a low molecular weight PEI.

18. The method of claim 17, wherein the PEI comprises PEI_{1.8k}.

19. The method of claim 18, wherein the PLGA/PEI_{1.8.k} particles are porous.

20. The method of any one of claims 13-18, wherein the PLGA/PEI particles are porous.

21. The method of any one of claims 13-20, wherein the pathological effect or symptom is selected from at least one of cardiac tissue damage; cardiomyocyte necrosis; contractile dysfunction; cardiac extracellular matrix remodeling; cardiomyocyte loss; apoptosis; necrosis; cardiac fibrosis; angiogenesis; hemodynamic and cardiac geometric deterioration in the heart, coronary artery, or vascular system; or systolic/diastolic dysfunction.

22. The method of any one of claims 13-21, wherein the administration of the stem cell-loaded PLGA/PEI particles is via at least one of an intramyocardial injection, a local injection, an intracoronary injection, or an intravenous injection.

23. The method of claim 22, wherein the particles are injected at or adjacent at least one of an infarct, a border zone of an infarct, or a central fibrotic zone of an infarct.

24. A poly(lactic-co-glycolic acid)/polyethylenimine (PLGA/PEI) particle, comprising:
PLGA; and
PEI_{1.8k}, wherein the particle has a diameter of about 100 μm to about 800 μm, wherein the particle comprises a plurality of pores, and wherein each pore has a diameter of up to about 20 μm.

25. The particle of claim 24, wherein the diameter of the particle is about 100 μm to about 300 μm.

26. The particle of claim 24 or claim 25, wherein the particles are loaded with stem cells.

27. A method of making poly(lactic-co-glycolic acid)/polyethylenimine (PLGA/PEI) particles, comprising the steps of:
combining poly(lactic-co-glycolic acid) (PLGA) with methylene chloride to generate a PLGA solution;
diluting polyethylenimine (PEI) in acetone to generate a PEI solution;
mixing a portion of the PLGA solution with a portion of the PEI solution to generate a pre-homogenization solution;
homogenizing the pre-homogenization solution to generate a primary emulsion;
combining the primary emulsion with a polyvinyl alcohol solution to generate a primary emulsion solution; and
re-emulsifying the primary emulsion solution to generate PLGA/PEI particles.

28. The method of claim 27, further comprising the step of:
adding a sodium chloride solution to a portion of the pre-homogenization solution.

29. The method of claim 28, wherein the PLGA/PEI particles are porous.

30. The method of any one of claims 27-29, wherein the PEI comprises a low molecular weight PEI.

31. The method of claim 30, wherein the PEI comprises PEI_{1.8k}.
